(19) United States
(12) Patent Application Publication
(10) Pub. No.: US 2025/0235091 A1
(43) Pub. Date: Jul. 24, 2025

(54) ENDOSCOPE, IMAGE PICKUP MODULE, AND MANUFACTURING METHOD OF ENDOSCOPE

(71) Applicant: OLYMPUS MEDICAL SYSTEMS CORP., Tokyo (JP)

(72) Inventor: Junya YAMADA, Tokyo (JP)

(73) Assignee: OLYMPUS MEDICAL SYSTEMS CORP., Tokyo (JP)

(21) Appl. No.: 19/025,194

(22) Filed: Jan. 16, 2025

Related U.S. Application Data
(60) Provisional application No. 63/622,834, filed on Jan. 19, 2024.

(51) Int. Cl. A61B 1/05; A61B 1/00

(52) U.S. Cl. CPC A61B 1/05; A61B 1/0008; A61B 1/0011

(57) ABSTRACT

In an insertion potion for use with an endoscope comprises a frame disposed in a distal end portion of the insertion portion; a three-dimensional wiring board fixed to the frame; and a camera. The three-dimensional wiring board comprises a first surface, a second surface arranged distally relative to the first surface, and a third surface arranged proximally relative to the first surface. The camera is mounted on the first surface inclined at a first angle relative to a longitudinal direction of the distal end portion. The frame includes a fourth surface contacting the second surface and a fifth surface contacting the third surface.

### CROSS-REFERENCE TO RELATED APPLICATION

This application is based on and claims priority under 35 U.S.C. § 119 to U.S. Provisional Application No. 63/622,834, filed on Jan. 19, 2024, the entire contents of which are incorporated herein by reference.

### FIELD OF DISCLOSURE

The present disclosure relates to an endoscope in which an image pickup module is disposed in a distal end portion of an insertion portion, the image pickup module disposed in the distal end portion of the insertion portion of the endoscope, and a manufacturing method of the endoscope in which the image pickup module is disposed in the distal end portion of the insertion portion.

### BACKGROUND

In an oblique endoscope, a direction in which an image pickup device picks up an image (observation direction) is inclined at a predetermined angle with respect to a longitudinal direction of a distal end portion of an insertion portion. The oblique endoscope cannot grasp a position of a treatment instrument that protrude from a side surface of the distal end portion to perform treatments, if a predetermined direction cannot be observed, for example.

WO2019/138606 discloses an oblique endoscope including an image pickup module in which an image pickup device is mounted on an inclined surface of a stacked substrate. The image pickup device is adhered and fixed to a lens unit via a cover glass. A lens holder of the lens unit is inserted and fixed into a through hole of a distal end frame. An optical axis direction of the lens unit, that is, an observation direction of the image pickup device is fixed in a direction of a center axis of the through hole. The center axis of the through hole is inclined at a predetermined angle with respect to a distal end axis of the endoscope.

### SUMMARY

An insertion potion for use with an endoscope of an embodiment of the present disclosure includes a frame disposed in a distal end portion of the insertion portion; a three-dimensional wiring board fixed to the frame; and a camera. The three-dimensional wiring board comprises a first surface, a second surface arranged distally relative to the first surface, and a third surface arranged proximally relative to the first surface. The camera is mounted on the first surface inclined at a first angle relative to a longitudinal direction of the distal end portion. The frame includes a fourth surface contacting the second surface and a fifth surface contacting the third surface.

A manufacturing method of an endoscope of the embodiment of the present disclosure includes fabricating a three-dimensional wiring board including a first surface, a second surface arranged distally relative to the first surface, and a third surface arranged proximally relative to the first surface, mounting a camera on the first surface, bringing the second surface in contact with a fourth surface of aa frame, and bringing the third surface in contact with a fifth surface of the frame, injecting an adhesive in a gap between the three-dimensional wiring board and the frame, and fixing the three-dimensional wiring board with mounted camera and cured adhesive to a distal end portion of an insertion portion of the endoscope.

### BRIEF DESCRIPTION OF THE DRAWINGS

FIG. 1 is a configuration view of an endoscope system including an endoscope of an embodiment of the present disclosure.

FIG. 2 is a perspective view of a distal end portion of the endoscope of the embodiment of the present disclosure.

FIG. 3 is a perspective cross-sectional view of the distal end portion of the endoscope of the embodiment of the present disclosure.

FIG. 4 is a perspective view of a three-dimensional wiring board of the endoscope of the embodiment of the present disclosure.

FIG. 5 is an exploded cross-sectional view of the distal end portion of the endoscope of the embodiment of the present disclosure.

FIG. 6 is a flowchart of a manufacturing method of the endoscope of the embodiment of the present disclosure.

FIG. 7 is a perspective cross-sectional view of a camera unit of the endoscope of the embodiment of the present disclosure.

FIG. 8 is a perspective cross-sectional view of the three-dimensional wiring board of the endoscope of the embodiment of the present disclosure.

FIG. 9 is a cross-sectional view for illustrating the manufacturing method of the endoscope of the embodiment of the present disclosure.

FIG. 10 is a cross-sectional view for illustrating the manufacturing method of the endoscope of the embodiment of the present disclosure.

FIG. 11 is a cross-sectional view of a distal end portion of an endoscope of a modification 1 of the embodiment of the present disclosure.

FIG. 12 is a cross-sectional view of a distal end portion of an endoscope of a modification 2 of the embodiment of the present disclosure.

FIG. 13 is a cross-sectional view of a distal end portion of an endoscope of a modification 3 of the embodiment of the present disclosure.

### DETAILED DESCRIPTION

### Embodiment

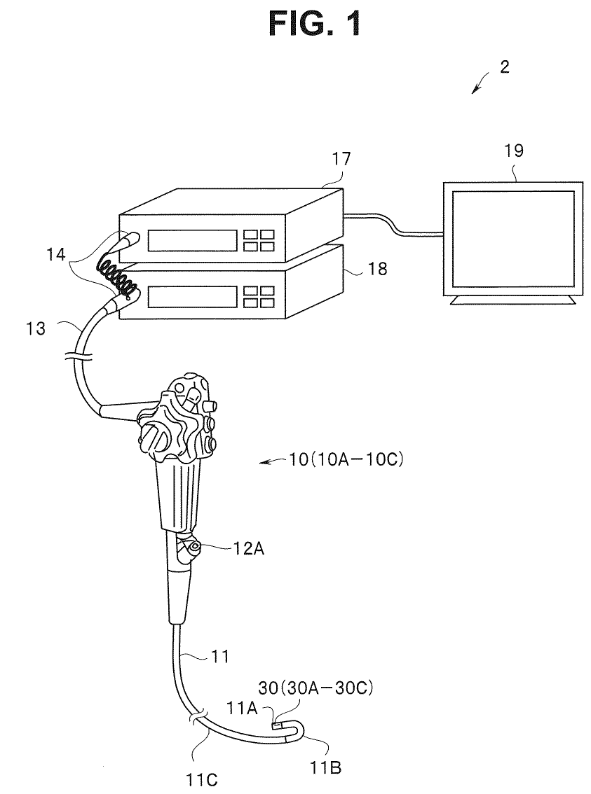

An endoscope system 2 including an endoscope 10 of an embodiment as shown in FIG. 1 includes the endoscope 10, a processor 17, an optical device 18, and a monitor 19.

In the following description, the drawings based on the embodiments are schematic illustrations. The relation between the thickness and the width of each portion, the ratio in thickness and the relative angle of each portions, and the like differ from the actual ones. There are also some portions with different dimensional relations and ratios among the drawings. Illustration and reference signs of part of the constituent elements will be omitted.

The endoscope 10 is an oblique endoscope in which an insertion portion 11 is configured to be inserted in a body of a subject to pick up an in-vivo image and output an image pickup signal. An operation portion 12 provided with various buttons for operating the endoscope 10 is disposed in a proximal end portion of the insertion portion 11 of the endoscope 10. The insertion portion 11 includes a distal end portion 11A in which an image pickup module 30 is disposed, a bending portion 11B that is continuously provided on a proximal end portion of the distal end portion 11A and is bendable, and a flexible portion 11C that is continuously provided on a proximal end portion of the bending portion 11B. The bending portion 11B is bent by operating the operation portion 12.

A universal cord 13 extended from the operation portion 12 is connected to the processor 17 and the optical device 18 via a connector 14. As will be described later, a treatment instrument is inserted from a treatment instrument insertion port 12A of the operation portion 12.

The processor 17 controls the entire endoscope system 2, and performs signal processing on an image pickup signal outputted by the image pickup module 30 to output the processed image pickup signal as an image signal. The monitor 19 displays the image signal outputted by the processor 17.

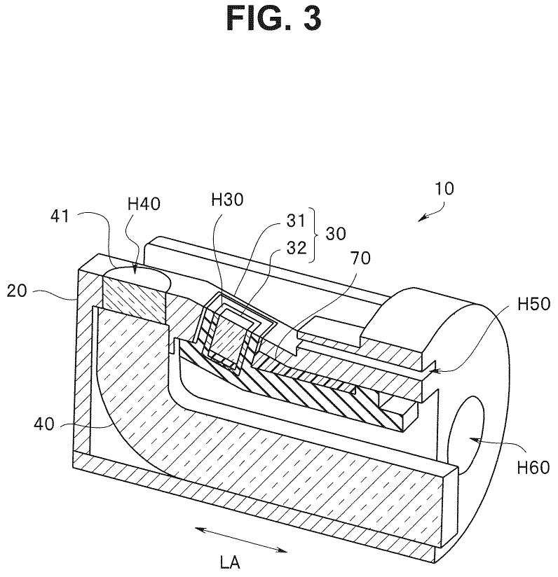

The optical device 18 includes a white LED, for example. An illumination light emitted from the optical device 18 is guided to the distal end portion 11A by a light guide 40 (see FIG. 3) inserted through the universal cord 13 and the insertion portion 11 and illuminates an object by way of an illumination lens 41 (see FIG. 3).

Note that the endoscope 10 is a medical flexible endoscope, but an endoscope of another embodiment may be a rigid endoscope or an industrial endoscope.

### Configuration of Distal End Portion

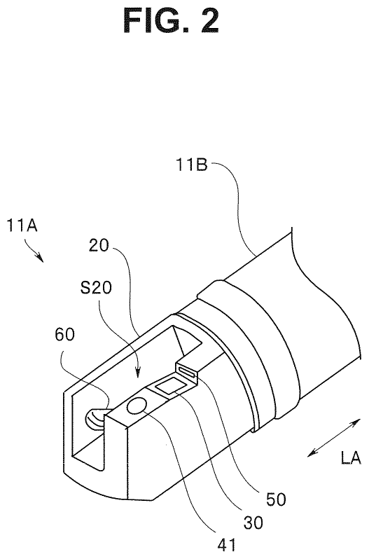

As shown in FIGS. 2 and 3, a distal end frame 20 which is a main member of the distal end portion 11A of the endoscope 10 is a rigid member made of metal, such as stainless steel. The bending portion 11B is continuously provided on a proximal end side of the distal end frame 20 arranged on a distal end of the insertion portion 11.

A raising base housing space S20 for housing a raising base (forceps elevator) 60 is formed in the distal end frame 20. Though not shown, the raising base 60 is used for operating a projecting direction of the treatment instrument that is inserted from the treatment instrument insertion port 12A and protrudes from an opening of the distal end portion 11A by way of a channel tube.

In the distal end frame 20, the illumination lens 41, the image pickup module 30, and a cleaning nozzle 50 are disposed in the order along a longitudinal direction LA of the distal end portion 11A. The illumination lens 41 emits an illumination light toward an object. The cleaning nozzle 50 ejects fluid for removing adhered substances towards the illumination lens 41 and the image pickup module 30.

Specifically, as shown in FIG. 3, the distal end frame 20 includes a through hole H60 to which the channel tube is connected, a through hole H30 in which the image pickup module 30 is inserted, a through hole H40 in which the illumination lens 41 is disposed, and a through hole H50 to which the cleaning nozzle 50 is connected. The through hole H30 includes openings on an outer surface and an inner surface of the distal end frame 20, for example. Though not shown, the distal end frame 20 has a large opening on a side surface, and after disposing a plurality of members inside thereof, the opening is blocked by a side surface lid.

The image pickup module 30 includes a three-dimensional wiring board 31 and a camera unit 32. As will be described later, the image pickup module 30 is fixed to the distal end frame 20 using an adhesive 70.

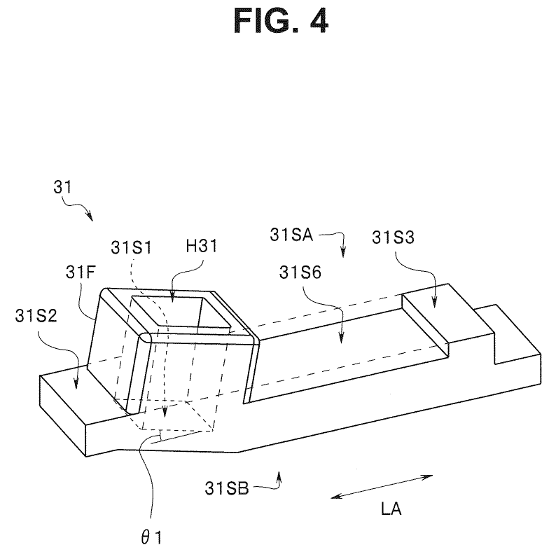

As shown in FIG. 4, the three-dimensional wiring board 31 has a complex three-dimensional structure. The three-dimensional wiring board 31 is an MID (molded interconnect device) in which a conductor pattern (not shown) is disposed on a surface of a three-dimensional molding that is injection-molded. The three-dimensional wiring board 31 allows a shape to have a function, and in addition, the conductor pattern to be formed on an inclined surface, a vertical surface, a curved surface, the through hole, and the like, unlike a flat wiring board.

An upper surface 31SA of the three-dimensional wiring board 31 includes a first surface 31S1, a second surface 31S2 on a distal end side of the first surface 31S1, a third surface 31S3 on a proximal end side of the first surface 31S1, and a sixth surface 31S6 between the first surface 31S1 and the third surface 31S3. The three-dimensional wiring board 31 includes a recess H31 that is surrounded by a frame-shaped wall 31F and includes the first surface 31S1 as a bottom surface. Though not shown, a plurality of wirings are disposed on a lower surface 31SB of the three-dimensional wiring board 31. The elongated three-dimensional wiring board 31 is fixed to the distal end frame 20 such that a longitudinal direction thereof is the same as the longitudinal direction LA of the distal end portion 11A. The sixth surface 31S6 can be larger than the third surface 31S3. The sixth surface 31S6 can have larger area than the third surface 31S3. The sixth surface 31S6 can have larger length than the third surface 31S3 in the longitudinal direction LA. The third surface 31S3 can be larger than the second surface 31S6. The third surface 31S3 can have larger area than second surface 31S6. The third surface 31S3 can have larger length than second surface 31S6.in the longitudinal direction LA. The longitudinal direction LA can be extending along at least one of a longitudinal axis of the insertion portion, a longitudinal axis of the distal end portion, the frame, three-dimensional wiring board 31.

The second surface 31S2 and the third surface 31S3 are located on the same plane and parallel to each other across the first surface 31S1. Meanwhile, the first surface 31S1 is inclined at a first angle θ1 with respect to the second surface 31S2 and the third surface 31S3. Each of the second and third surfaces 31S2 and 31S32 can have the first angle θ1 relative to the first surface 31S1, the first surface 31S1 can be located between the second and third surfaces 31S2 and 31S32 in the longitudinal direction LA. The parallel can include a manufacturing error within ±7 degrees, for example.

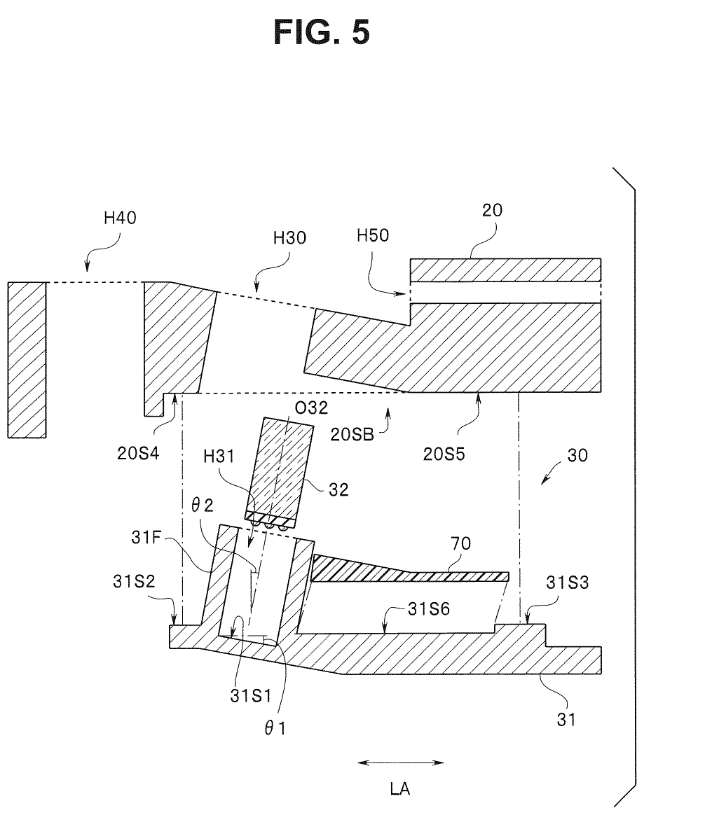

As shown in an exploded view of FIG. 5, the camera unit 32 is inserted in the recess H31 of the three-dimensional wiring board 31 of the image pickup module 30. Specifically, the camera unit 32 is mounted on the first surface 31S1 of the three-dimensional wiring board 31. An optical axis O32 of the camera unit 32 is perpendicular to the first surface 31S1. Therefore, a second angle θ2 formed between the optical axis O32 of the camera unit 32 and a line orthogonal to the longitudinal direction LA is the same as the first angle θ1 which is an inclined angle of the first surface 31S1. The second angle θ2 is an angle of the optical axis O32 of the camera unit 32 in a direction orthogonal to the longitudinal direction LA.

The second angle θ2 is between −30 degrees and 30 degrees, for example. To ensure that the camera unit 32 is capable of observing in a predetermined direction, a manufacturing error of the second angle θ2 should be managed within ±7 degrees, for example.

In conventional endoscopes in which the lens holder of the lens unit is inserted and fixed in the through hole of the distal end frame described in the background art, there is a gap between an outer surface of the lens holder and an inner surface of the through hole. Therefore, the optical axis of the lens unit could be inclined away from a central axis of the through hole, making it impossible to observe a field of view in a predetermined direction.

The embodiment of the present disclosure aims to provide an oblique endoscope that is capable of observing in a predetermined direction, an image pickup module of the oblique endoscope that is capable of observing in the predetermined direction, and a manufacturing method of the oblique endoscope that is capable of observing in the predetermined direction.

As already described above, in the endoscope 10 (image pickup module 30), the second surface 31S2 and the third surface 31S3 of the three-dimensional wiring board 31 can be located on the same plane (one plane) and/or parallel to each other. Note that the first angle θ1 formed between each of the second surface S31S2 and the third surface 31S3 and the first surface 31S1 is determined at the time of designing the three-dimensional wiring board 31. The fourth surface 20S4 and the fifth surface 20S5 can be located on the same plane (one plane) and/or parallel to each other.

Meanwhile, a fourth surface 20S4 and a fifth surface 20S5 of an inner surface 20SB of the distal end frame 20 can be parallel to the longitudinal direction LA of the distal end portion 11A. The three-dimensional wiring board 31 is fixed to the inner surface 20SB of the distal end frame 20 using the adhesive 70 in a state in which the second surface 31S2 and the third surface 31S3 are in contact with the fourth surface 20S4 and the fifth surface 20S5 of the distal end frame 20. In this state, the wall 31F constituting the recess H31 of the three-dimensional wiring board 31 is inserted in the through hole H30 of the distal end frame 20.

The second surface 31S2 and the third surface 31S3 of the three-dimensional wiring board 31 are in contact with the fourth surface 20S4 and the fifth surface 20S5 of the distal end frame 20. Since the fourth surface 20S4 and the fifth surface 20S5 can be parallel to the longitudinal direction LA, the second surface 31S2 and the third surface 31S3 of the three-dimensional wiring board 31 are also naturally parallel to the longitudinal direction LA. Therefore, the first surface 31S1 inclined at the first angle θ1 with respect to the second surface 31S2 of the three-dimensional wiring board 31 is inclined at the first angle θ1 with respect to the longitudinal direction LA. The optical axis O32 of the camera unit 32 is inclined at the second angle θ2 equal to the first angle θ1 with respect to the longitudinal direction LA.

An angle of the image pickup module 30 with respect to the distal end frame 20 is defined by two surfaces (the second surface 31S2 on the distal end side and the third surface 31S3 on a rear end side) arranged sandwiching the first surface 31S1 on which the camera unit 32 is mounted. Therefore, in the endoscope 10, an angle of the optical axis O32 of the camera unit 32 with respect to the distal end frame 20, that is, an angle of the optical axis O32 with respect to the longitudinal direction LA is ensured to be highly accurate compared with an endoscope in which an angle between an image pickup module and a distal end frame is defined by one surface.

As described above, the image pickup module 30 of the embodiment includes the camera unit 32 and the three-dimensional wiring board 31. The three-dimensional wiring board 31 includes the first surface 31S1 on which the camera unit 32 is mounted, and the second surface 31S2 and the third surface 31S3 each having the first angle θ1 with respect to the first surface 31S1. The second surface 31S2 is located at one end side of the three-dimensional wiring board 31 in the longitudinal direction LA, and the third surface 31S3 is located on an opposite side of the second surface 31S2 across the camera unit 32. The second surface 31S2 and the third surface 31S3 are arranged to be respectively in contact with the two surfaces of the distal end frame 20 of the endoscope 10.

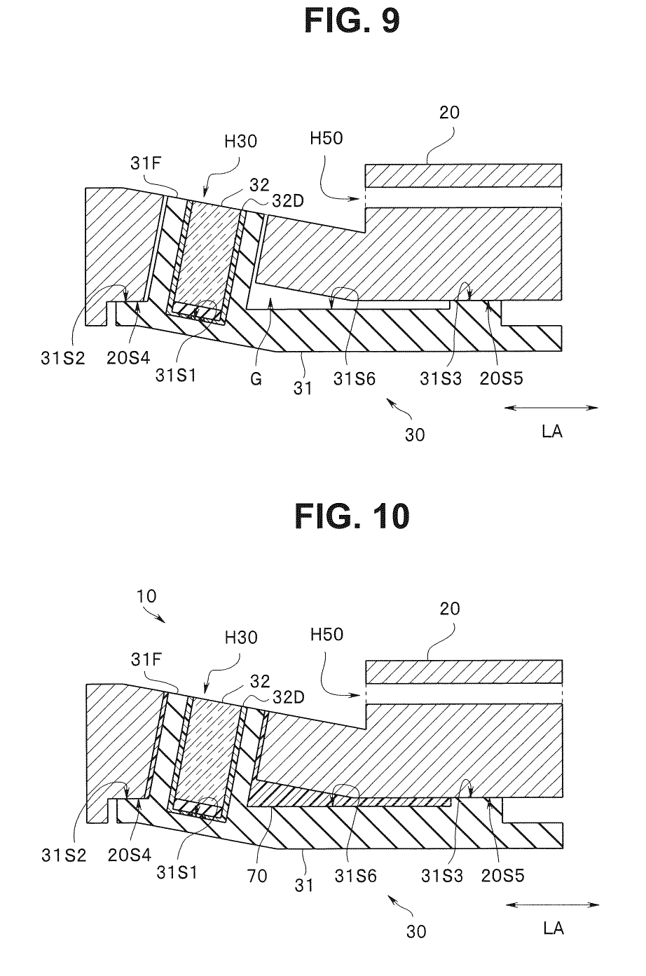

The adhesive 70 is disposed in a gap G (see FIG. 9) between the sixth surface 31S6 of the three-dimensional wiring board 31 and the distal end frame 20. The adhesive 70 is not disposed between the second surface 31S2 and the third surface 31S3 and the inner surface 20SB (the forth surface 20S4, the fifth surface 20S5) of the distal end frame 20. The gap G may be provided at other position between the three-dimensional wiring board 31 and the distal end frame 20 and the adhesive 70 may be disposed in the gap G.

Since the adhesive is not disposed on a contact surface for positioning (angle determining), the angle of the image pickup module 30 with respect to the distal end frame 20 may not cause variations due to variations in a thickness of the adhesive. For this reason, the endoscope 10 has particularly small manufacturing errors, and the accuracy of an inclined angle of the optical axis O32 of the camera unit 32 with respect to the longitudinal direction LA is within ±3 degrees, for example.

The embodiment of the present disclosure can provide the endoscope that is capable of observing in the predetermined direction, the image pickup module of the endoscope that is capable of observing in the predetermined direction, and the manufacturing method of the endoscope that is capable of observing in the predetermined direction.

### Manufacturing Method of Endoscope

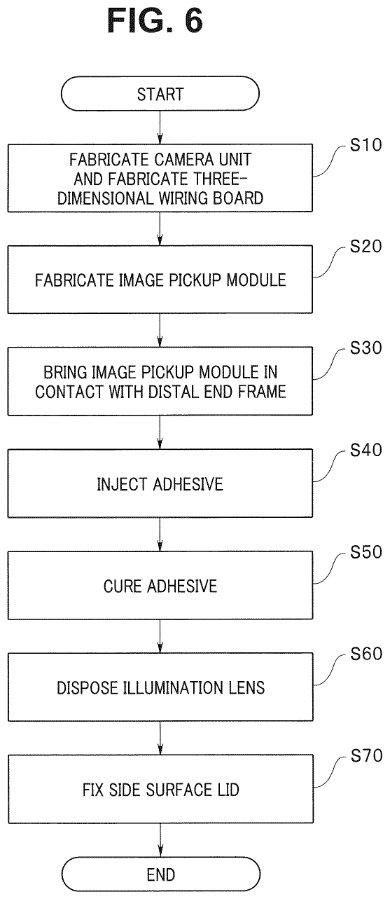

A manufacturing method of the endoscope 10, specifically, a manufacturing method of the distal end portion 11A will be described according to a flowchart of FIG. 6.

### <Step S10> Fabricating Camera Unit and Three-Dimensional Wiring Board

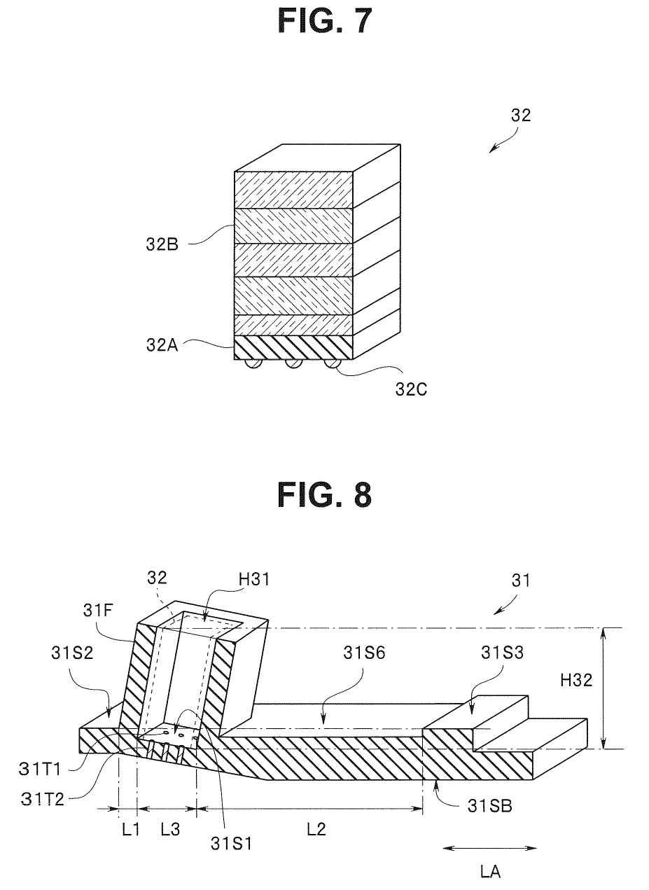

As shown in FIG. 7, the camera unit 32 includes an image pickup device 32A and an image pickup optical system 32B. The image pickup device 32A is an image sensor, such as CCD, a CMOS, or the like that converts an object image condensed by the image pickup optical system 32B into electric signals. The image pickup device 32A may include a cover glass. The image pickup optical system 32B includes a plurality of optical elements (lens, filter, or the like). The image pickup optical system 32B is fabricated by cutting a stacked wafer in which a plurality of optical wafers each including the plurality of optical elements are stacked.

A ball grid array including a plurality of bonding members 32C is disposed on a rear surface of the image pickup device 32A. The bonding member 32C is a solder ball, a gold bump, or the like.

As shown in FIG. 8, a plurality of pads 31T1 are disposed on the first surface 31S1 which is a bottom surface of the recess H31 of the three-dimensional wiring board 31. The three-dimensional wiring board 31 includes through-hole wirings 31T2 below the pads 31T1 to the lower surface 31SB on an opposite side of the first surface 31S1. The pads 31T1 and the through-hole wirings 31T2 are disposed by plating, screen printing, sputtering, metal deposition, or the like. Though not shown, wirings extended from the through-hole wirings 31T2 are disposed on the lower surface 31SB, an electronic component such as a chip condenser is mounted on the wirings, and a cable is further bonded to end portions thereof.

A first length L1 between the first surface 31S1 and the second surface 31S2 in the longitudinal direction LA is shorter than a second length L2 between the first surface 31S1 and the third surface 31S3 in the longitudinal direction LA. By reducing a length from the camera unit 32 to a distal end of the distal end frame 20, the endoscope 10 becomes smaller and less invasive.

Furthermore, in the direction orthogonal to the longitudinal direction LA, positions of the second surface 31S2 and the third surface 31S3 are within a range of a depth H32 (a length of the camera unit 32 in an optical axis direction) of the recess H31. For this reason, a length (an outer diameter) of the distal end portion 11A of the endoscope 10 in the direction orthogonal to the longitudinal direction LA is short and the endoscope 10 is less invasive. In the direction orthogonal to the longitudinal direction, the second and third surfaces 31S2 and 31S3 can be located closer to the frame than the first surface 31S1. In the direction orthogonal to the longitudinal direction, the second and third surfaces 31S2 and 31S3 can be located closer to an inner surface of the frame than the first surface 31S1. In the direction orthogonal to the longitudinal direction, the second and third surfaces 31S2 and 31S3 can be located further from a longitudinal axis of the distal end portion of the endoscope.

### <Step S20> Fabricating Image Pickup Module

The camera unit 32 is inserted in the recess H31 of the three-dimensional wiring board 31 and the bonding members 32C of the camera unit 32 and the pads 31T1 on the first surface 31S1 of the three-dimensional wiring board 31 are bonded. A sealing resin 32D is filled in a gap between the camera unit 32 housed in the recess H31 and wall surfaces of the recess H31. An electronic component such as a chip condenser is mounted on the wirings on the lower surface 31SB of the three-dimensional wiring board 31, and a cable is further bonded to end portions of the wirings. The three-dimensional wiring board 31 can include the recess H31 surrounded by the wall H31, the recess H31 includes the first surface 31S1 as a bottom surface. The camera can include the optical system 32B and the image sensor 32A. The camera can be housed in the recess, and the wall H31 can be inserted in a through hole of the frame 20. In a direction orthogonal to the longitudinal direction LA, the second surface 31S2 and the third surface 31S3 can be positioned between a top surface of the wall H31 and the first surface 31S1.

<Step S30> Bringing Image Pickup Module in Contact with Distal End Frame

As shown in FIG. 9, the three-dimensional wiring board 31 is temporarily fixed to be in contact with the inner surface of the distal end frame 20. Specifically, the second surface 31S2 of the three-dimensional wiring board 31 is brought in contact with the fourth surface 20S4 of the distal end frame 20, and the third surface 31S3 of the three-dimensional wiring board 31 is brought in contact with the fifth surface 20S5 of the distal end frame 20. The gap G is formed between the sixth surface 31S6 of the three-dimensional wiring board 31 and the distal end frame 20. The gap G may be provided in a different position from the above, as long as it is between the three-dimensional wiring board 31 and the distal end frame 20.

### <Step S40> Injecting Adhesive

As shown in FIG. 10, the adhesive 70 can be injected in the gap G between the sixth surface 31S6 of the three-dimensional wiring board 31 and the distal end frame 20. The adhesive 70 is a UV-curable, thermo-curable, or UV-thermal combination-curable epoxy resin, for example. The adhesive 70 is also disposed in a gap between the through hole H30 and the image pickup module 30.

The adhesive 70 is not disposed between the second surface 31S2 and the fourth surface 20S4 and between the third surface 31S3 and the fifth surface 20S5. However, it goes without saying that a small amount of the adhesive 70 may be present between the two surfaces in contact with each other, due to unavoidable gaps between the two surfaces caused by manufacturing errors or scratches on a face of the surface. The adhesive 70 may not be disposed between the second surface 31S2 and the fourth surface 20S4 of the frame 10, and the adhesive 70 may not be disposed between the third surface 31S3 and corresponding portions the fifth surface 20S5 of the frame 10.

### <Step S50> Curing Adhesive

The adhesive 70 is subjected to a curing treatment and the image pickup module 30 is fixed to the distal end frame 20.

### <Step S60> Disposing Illumination Lens

The illumination lens 41, the light guide 40 and the like are fixed to the distal end frame 20. The illumination lens 41 and the like may be fixed to the distal end frame 20 before the image pickup module 30 is fixed to the distal end frame 20.

### <Step S70> Fixing Side Surface Lid

A not shown side surface opening of the distal end frame 20 is blocked by fixing the side surface lid, for example. The above side surface opening may be blocked by fixing the side surface lid before the image pickup module 30 is fixed to the distal end frame 20.

According to the manufacturing method of the endoscope of the present embodiment, the endoscope that can accurately observe in the predetermined direction can be easily manufactured. For example, an endoscope can be easily manufactured with an accuracy of the inclined angle of the optical axis O32 of the camera unit 32 with respect to the longitudinal direction LA within ±3 degrees. The manufacturing method of the endoscope can comprise: fabricating the three-dimensional wiring board 31; mounting the camera 32 on the first surface 31S1; fabricating the frame 20 comprising fourth and fifth surfaces 20S4 and 20S5; bringing the second surface 31S2 in contact with the fourth surface 20S4 of the frame 20, and bringing the third surface 31S3 in contact with the fifth surface 20S4 and 20S5 of the frame 20; injecting the adhesive 70 in the gap G between the three-dimensional wiring board 31 and the frame 20; curing the adhesive 70; and fixing the three-dimensional wiring board 31 with mounted camera 32 and cured adhesive 70 to the distal end portion of the insertion portion of the endoscope. The three-dimensional wiring board 31 can comprise the first surface 31S1, the second surface 31S2 arranged distally relative to the first surface 31S1, and the third surface 31S3 arranged proximally relative to the first surface 31S1. The second and third surfaces are arranged across the first surface 31S1 and each of the second and third surfaces having the first angle relative to the first surface 31S1.

### Modification

Endoscopes 10A to 10C (image pickup modules 30A to 30C) of modifications are similar to the endoscope 10 and have the same effects as the effects of the endoscope 10. Therefore, in the following description, the constituent elements having the same functions as the endoscope 10 will be assigned with the same reference signs, and the description will be omitted.

### Modification 1

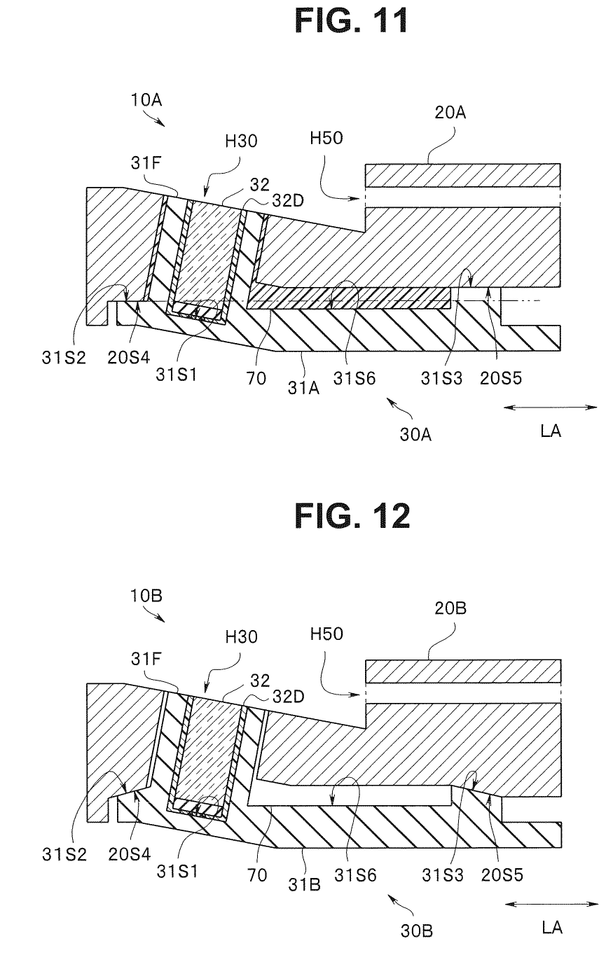

In the endoscope 10A of the present modification as shown in FIG. 11, a second surface 31S2 and a third surface 31S3 of a three-dimensional wiring board 31A of the image pickup module 30A are parallel to each other, but are not located on the same plane. Specifically, the third surface 31S3 is located on an upper side with respect to the second surface 31S2 in the drawing. Furthermore, a fourth surface 20S4 and a fifth surface 20S5 of a distal end frame 20A are parallel to each other, but are not located on the same plane.

However, the first surface 31S1 is inclined at a predetermined angle with respect to the longitudinal direction LA, with the second surface 31S2 in contact with the fourth surface 20S4 and the third surface 31S3 in contact with the fifth surface 20S5. Therefore, the optical axis 032 of the camera unit 32 is inclined at the predetermined angle with respect to the longitudinal direction LA.

### Modification 2

In the endoscope 10B of the present modification as shown in FIG. 12, a second surface 31S2 and a third surface 31S3 of a three-dimensional wiring board 31B of the image pickup module 30B are not parallel to each other, and are not parallel to the longitudinal direction LA. Furthermore, a fourth surface 20S4 and a fifth surface 20S5 of a distal end frame 20B are not parallel to each other, and are not parallel to the longitudinal direction LA. At least one of the second surface 31S2 or third surface 31S3 can be inclined at a second angle relative to the longitudinal direction of the distal end portion. The second angle can be different from the first angle. The second angle can be the same as the first angle.

However, the second surface 31S2 of the three-dimensional wiring board 31B is parallel to the fourth surface 20S4 of the distal end frame 20B, and the third surface 31S3 is parallel to the fifth surface 20S5. The first surface 31S1 is inclined at a predetermined angle with respect to the longitudinal direction LA, with the second surface 31S2 in contact with the fourth surface 20S4 and the third surface 31S3 in contact with the fifth surface 20S5. Therefore, the optical axis O32 of the camera unit 32 is inclined at the predetermined angle with respect to the longitudinal direction LA.

### Modification 3

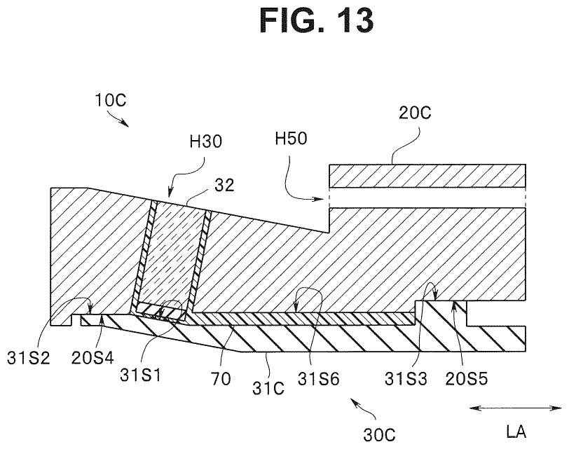

In the endoscope 10C of the present modification as shown in FIG. 13, a three-dimensional wiring board 31C of the image pickup module 30C does not include a recess surrounded by a wall as the three-dimensional wiring board 31 does. An inner diameter of a through hole H20 of a distal end frame 20C is slightly larger than an outer diameter of the camera unit 32.

The endoscope 10C includes no walls forming a recess, thus reducing a length of the distal end portion. Furthermore, the endoscope 10C can accurately observe in a predetermined direction as the endoscope 10 does.

Note that the above described range of numerical values is not limited to the above described range and can be increased or decreased as appropriate. Furthermore, the endoscope 10 may be a rigid endoscope whose insertion portion 11 is rigid. The present disclosure is not limited to the aforementioned embodiments and the like, and various changes, modification, or the like are available within the scope without departing from the gist of the present disclosure.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

Example 1. An endoscope comprising:
an insertion potion including a distal end frame in a distal end portion of the insertion portion, an image pickup module being fixed to the distal end frame, wherein
the image pickup module includes a camera unit including an image pickup optical system and an image pickup device, and a three-dimensional wiring board,
the three-dimensional wiring board includes a first surface, a second surface on a distal end side of the first surface, and a third surface on a proximal end side of the first surface, and the camera unit is mounted on the first surface inclined at a first angle with respect to a longitudinal direction of the distal end portion, and
the distal end frame includes a fourth surface in contact with the second surface and a fifth surface in contact with the third surface.

Example 2. The endoscope according to Example 1, wherein the second surface, the third surface, the fourth surface, and the fifth surface are parallel to the longitudinal direction.

Example 3. The endoscope according to Example 1, wherein the three-dimensional wiring board is an MID.

Example 4. The endoscope according to Example 3, wherein
the three-dimensional wiring board includes a recess that is surrounded by a wall and includes the first surface as a bottom surface,
the camera unit is housed in the recess, and
the wall constituting the recess is inserted in a through hole of the distal end frame.

Example 5. The endoscope according to Example 1, wherein a first length between the first surface and the second surface is shorter than a second length between the first surface and the third surface.

Example 6. The endoscope according to Example 1, wherein the first angle is equal to a second angle formed between an optical axis of the camera unit and a perpendicular of the first surface.

Example 7. The endoscope according to Example 1, wherein the second surface and the third surface are located on one plane.

Example 8. The endoscope according to Example 7, wherein the fourth surface and the fifth surface are located on the one plane.

Example 9. The endoscope according to Example 7, wherein in a direction orthogonal to the longitudinal direction, positions of the second surface and the third surface are within a range of a length of the camera unit in an optical axis direction.

Example 10. The endoscope according to Example 1, wherein
the three-dimensional wiring board includes a sixth surface between the first surface and the third surface, and
an adhesive is disposed in a gap between the sixth surface and the distal end frame.

Example 11. The endoscope according to Example 10, wherein the adhesive is not disposed between the second surface and the third surface and the distal end frame.

Example 12. An image pickup module comprising:
a camera unit; and
a three-dimensional wiring board including a first surface on which the camera unit is mounted, and a second surface and a third surface each having a first angle with respect to the first surface, the second surface being located on one end side of the three-dimensional wiring board in a longitudinal direction, the third surface being located on an opposite side of the second surface across the first surface, and the second surface and the third surface being arranged to be respectively in contact with two surfaces of a distal end frame of an endoscope.

Example 13. A manufacturing method of an endoscope comprising:
fabricating a three-dimensional wiring board including a first surface, a second surface on a distal end side of the first surface, and a third surface on a proximal end side of the second first surface, the second surface and the third surface that are arranged across the first surface each having a first angle with respect to the first surface;
mounting a camera unit on the first surface of the three-dimensional wiring board to fabricate an image pickup module;
fabricating a distal end frame, the distal end frame including a fourth surface and a fifth surface;
bringing the second surface of the three-dimensional wiring board in contact with the fourth surface of the distal and frame, and bringing the third surface of the three-dimensional wiring board in contact with the fifth surface of the distal end frame;
injecting an adhesive in a gap between the three-dimensional wiring board and the distal end frame;
subjecting the adhesive to a curing treatment; and fixing the image pickup module to a distal end portion of an insertion portion of an endoscope.

Example 14. The manufacturing method of the endoscope according to Example 13, wherein
the three-dimensional wiring board includes a recess that includes the first surface as a bottom surface and is surrounded by a wall, and
the recess houses the camera unit including an image pickup optical system and an image pickup device.

What is claimed is:

1. An insertion potion for use with an endoscope comprising:
a frame disposed in a distal end portion of the insertion portion;
a three-dimensional wiring board fixed to the frame, the three-dimensional wiring board comprising:
a first surface;
a second surface arranged distally relative to the first surface; and
a third surface arranged proximally relative to the first surface,
a camera mounted on the first surface inclined at a first angle relative to a longitudinal direction of the distal end portion, and
wherein the frame includes a fourth surface contacting the second surface and a fifth surface contacting the third surface.

2. The insertion potion according to claim 1, wherein the second surface, the third surface are parallel to the longitudinal direction.

3. The insertion potion according to claim 2, wherein the fourth surface, and the fifth surface are parallel to the longitudinal direction.

4. The insertion potion according to claim 1, wherein the three-dimensional wiring board is an MID.

5. The insertion potion according to claim 1, wherein
the three-dimensional wiring board includes a recess surrounded by a wall, the recess includes the first surface as a bottom surface,
the camera includes an optical system and an image sensor,
the camera is housed in the recess, and
the wall is inserted in a through hole of the frame.

6. The insertion potion according to claim 5, wherein in a direction orthogonal to the longitudinal direction, the second surface and the third surface are positioned between a top surface of the wall and the first surface.

7. The insertion potion according to claim 1, wherein a first length between the first surface and the second surface is shorter than a second length between the first surface and the third surface.

8. The insertion potion according to claim 1, wherein the first angle is equal to a second angle formed between an optical axis of the camera and a line perpendicular to the longitudinal direction.

9. The insertion potion according to claim 1, wherein the second surface and the third surface are located on one plane.

10. The insertion potion according to claim 9, wherein the fourth surface and the fifth surface are located on the one plane.

11. The insertion potion according to claim 1, wherein
the three-dimensional wiring board includes a sixth surface between the first surface and the third surface, and
an adhesive is disposed in a gap between the sixth surface and a corresponding portion of the frame.

12. The insertion potion according to claim 11, wherein the adhesive is not disposed between the second surface and the fourth surface of the frame, and
the adhesive is not disposed between the third surface and the fifth surface of the frame.

13. The insertion potion according to claim 1, wherein
the three-dimensional wiring board includes a sixth surface between the first surface and the third surface, and
the sixth surface is larger than the third surface.

14. The insertion potion according to claim 1, wherein in a direction orthogonal to the longitudinal direction, the second and third surfaces are located closer to the frame than the first surface.

15. The insertion potion according to claim 1, wherein at least one of the second surface or third surface is inclined at a second angle relative to a longitudinal direction of the distal end portion.

16. The insertion potion according to claim 15, wherein the second angle is different from the first angle.

17. An endoscope comprising:
the insertion portion according to claim 1.

18. An image pickup module comprising:
a camera; and
a three-dimensional wiring board comprising:
a first surface on which the camera is mounted;
a second surface; and
a third surface,
wherein each of the second and third surfaces having a first angle relative to the first surface, the first surface located between the second and third surfaces in a longitudinal direction, and
the second and third surfaces contacting fourth and fifth surfaces, respectively, of a frame of an endoscope.

19. A manufacturing method of an endoscope comprising:
fabricating a three-dimensional wiring board comprising:
a first surface;
a second surface arranged distally relative to the first surface; and
a third surface arranged proximally relative to the first surface,
wherein each of the second and third surfaces having a first angle relative to the first surface,
mounting a camera on the first surface;
bringing the second surface in contact with a fourth surface of a frame, and bringing the third surface in contact with a fifth surface of the frame;
injecting an adhesive in a gap between the three-dimensional wiring board and the frame; and
fixing the three-dimensional wiring board with mounted camera and cured adhesive to a distal end portion of an insertion portion of the endoscope.

20. The manufacturing method according to claim 19, wherein
the three-dimensional wiring board includes a recess including the first surface as a bottom surface, the first surface being surrounded by a wall, and
the recess houses the camera including an optical system and an image sensor.
